(19) United States
(12) Plant Patent Application Publication
(10) Pub. No.: US 2007/0226860 P1
(43) Pub. Date: Sep. 27, 2007

(54) Variety of Begonia plant named 'Solenia Super Soft Pink'

(75) Inventor: Christiane Heins, Hann. Munden (DE)

Correspondence Address: THE WEBB LAW FIRM, P.C., 700 KOPPERS BUILDING, 436 SEVENTH AVENUE, PITTSBURGH, PA, 15219, US

(73) Assignee: Ernst Benary Samenzucht GmbH, Hann. Munden (DE)

(21) Appl. No.: 11/387,328

(22) Filed: Mar. 23, 2006

(51) Int. Cl. A01H 5/00

(52) U.S. Cl. PLT/347

(57) ABSTRACT

A new and distinct Begonia plant having long-lasting vibrant pink flowers that exhibits good outdoor and garden performance.

### BOTANICAL CLASSIFICATION

Begonia elatior hybrid [Begonia×hiemalis]

### VARIETAL DENOMINATION

‘Solenia Super Soft Pink’

### BACKGROUND OF THE INVENTION

The present invention comprises a new and distinct cultivar of Begonia plant known by the varietal name ‘Solenia Super Soft Pink’. The new variety was discovered in 1998 in a selected breeding program in Gimte, Germany. The purpose of the breeding program was to develop a new variety with high sun and rain resistance, as well as good garden and outdoor performance. The new variety is the result of a cross between STHR 47/412 (female parent) and TS 24/124 (male parent). The new variety differs in flower color, habit and foliage characteristics from both parents. The new variety was first asexually reproduced in the winter 1998/1999 season by cuttings in Germany. The new variety has been trial and field tested in Germany and has been found to retain its distinctive characteristics and remain true to type through successive propagations.

The following traits distinguish ‘Solenia Super Soft Pink’ as a new and distinct cultivar:
1. The flower color is light pink; and
2. The new variety exhibits good outdoor and garden performance.

### DESCRIPTION OF THE DRAWINGS

The accompanying photographic drawings illustrate the new variety, with the color being as nearly true as is possible with color illustrations of this type:

FIG. 1 is photograph of an entire plant of the new variety; and

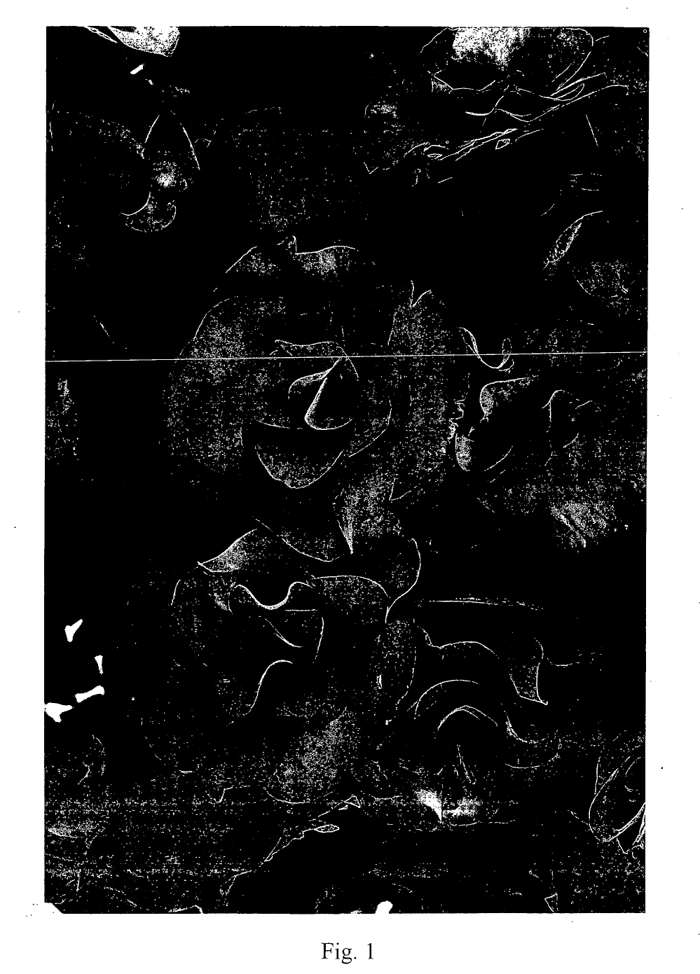

FIG. 2 is a close-up photograph of the flowers of the new variety.

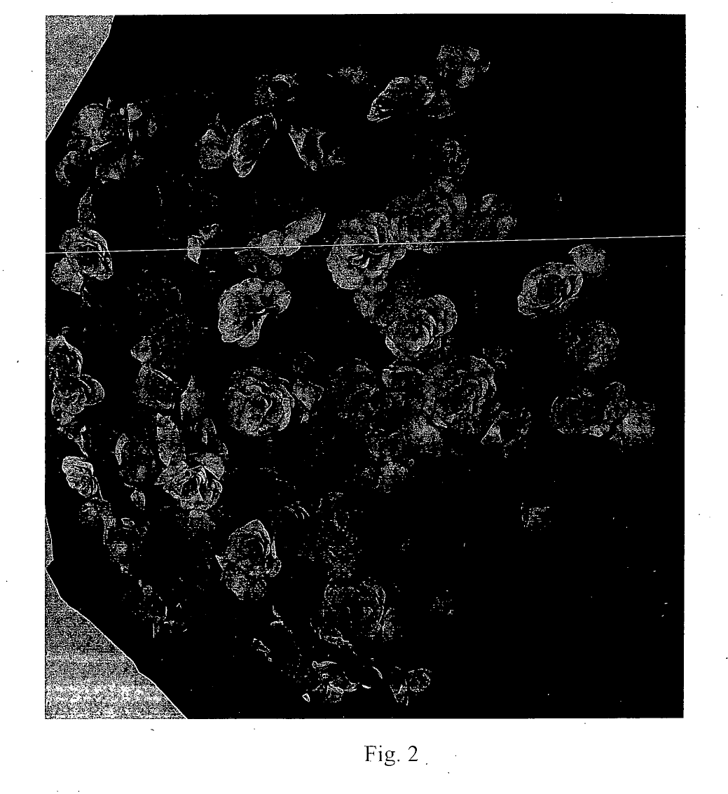

### DESCRIPTION OF THE PLANT

The following detailed description sets forth characteristics of the new cultivar. The data which defines these characteristics were collected by asexual reproductions by cuttings carried out in Germany. The plants were exposed to an average temperature of 18° C. and 14 hours of long day light. Plants for the description were 6 months old and in 13 cm containers. The color readings were taken indoors under natural light. Color references are primarily to the R.H.S. Colour Chart of The Royal Horticultural Society of London.
Plant:
   Time to initiate roots.—Two weeks at 20° C.
   Time to develop roots.—Four weeks at 20° C.
   Form.—Upright.
   Height from soil to top of flowers.—9.84″ (25 cm).
   Plant diameter.—13.39″ (34 cm).
   Vigor.—Good.
   Branching habit.—Medium.
   Lateral branches.—Color: Light green. Texture: Smooth. Length: 7.09″ (18 cm). Diameter: 6.3″ (16 cm). Internode length: 0.98″ (2.5 cm). Strength: 0.16″ (0.4 cm). Quantity: 5.
   Foliage.—Arrangement: Alternate. Number of leaves per stem: 4 to 5. Size of leaf: Length: 5.5″ (14 cm). Width: 4.3″ (11 cm). Shape of leaf: Typical Begonia leaf shape. Shape of apex: Acute. Shape of base: Slightly overlapping. Texture: Smooth, with venation present. Margin type: Bicrenate. Fragrance: Medium. Color: Young and mature leaves: Upper surface: Green Group 139A. Lower surface: Green Group 139C. Petiole: Length: 1.57″ (4 cm). Diameter: 0.12″ (0.3 cm). Color: Yellow-Green Group 145B. Texture: Very small pubescence present. Veins: Venation pattern: Star-shaped. Color: Upper surface: Green Group 137C. Lower surface: Green Group 137D.
Flower:
   Natural flowering season.—May/June in Germany.
   Flower habit.—Multiflora, double flowering.
   Number of flowers per plant.—60-80.
   Time of day of flower opening.—Flowers are always open.
   Longevity.—3-4 weeks on the plant.
   Flower height.—0.79″ (2 cm).
   Flower diameter.—2.36″ (6 cm).
   Bud.—Stage of development when described: At the beginning of opening. Shape: Semi-circle. Diameter: 1.57″ (4 cm). Length: 0.98″ (2.5 cm). Color: Red-Purple Group 61D.
   Petals.—Arrangement: Double. Quantity: 25-30. Length: 1.18″ (3 cm). Width: 1.57″ (4 cm). Shape: Semi-circle. Apex: Round. Base: Even and straight. Margin: Entire. Texture: Slightly grooved. Color: When opening and fully opened: Red-Purple Group 61D.
   Peduncles.—Length: 2.76″-3.54″ (7-9 cm). Diameter: 0.16″ (0.4 cm). Color: Green Group 143B. Texture: Smooth. Orientation: Upright. Strength: Strong.
   Pedicels.—Length: 0.39″ (1 cm). Width: 0.08″ (0.2 cm). Color: Yellow-Green Group 146B. Strength: Strong. Orientation: Angled.
   Disease resistance.—No unusual susceptibility noticed to date.
   Weather tolerance.—Good.
   Style color.—Greyed-Orange Group 168D.
   Stigma.—Shape: Typical for varieties of this type. Color: Orange Group 28A. Ovary color: Light green.

I claim:

1. A new and distinct variety of Begonia plant substantially as shown and described.
